(19) United States
(12) Patent Application Publication
(10) Pub. No.: US 2026/0306854 A1
(43) Pub. Date: Oct. 8, 2026

(54) Electrophysiology System With Mapping Stimulation

(71) Applicant: Reach Neuro, Inc., Providence, RI (US)

(72) Inventors: Douglas Weber, Pittsburgh, PA (US); Luigi Borda, Pittsburgh, PA (US); Marc Philip Powell, Bristol, RI (US); Marco Capogrosso, Pittsburgh, PA (US)

(21) Appl. No.: 19/640,080

(22) Filed: Apr. 6, 2026

Related U.S. Application Data
(60) Provisional application No. 63/784,527, filed on Apr. 7, 2025.

(51) Int. Cl. A61N 1/372; A61N 1/05; A61N 1/36

(52) U.S. Cl. CPC A61N 1/372; A61N 1/0551; A61N 1/36003; A61N 1/36135; A61N 1/3615; A61N 1/36171; A61N 1/36175; A61N 1/36178; A61N 1/36185

(57) ABSTRACT

A method of adjusting placement of an electrode of a spinal cord stimulation device in a subject is disclosed. The method comprises implanting the electrode proximate to a dorsal root of a spinal cord. A series of electrical stimulation pulses are delivered to the electrode. Evoked compound muscle action potentials (CMAP) may be recorded from a plurality of muscles of the subject. The recorded evoked CMAPs may be processed to generate a muscle map associating the electrode with corresponding muscle activation data. An indication for adjusting the placement of the electrode may be generated based on the muscle map. The placement of the electrode may be adjusted according to the indication.

### CROSS-REFERENCE TO RELATED APPLICATIONS

This application claims the benefit of U.S. Provisional Patent Application No. 63/784,527, filed Apr. 7, 2025, the entire contents of which are incorporated herein by reference.

### TECHNICAL FIELD

This disclosure relates to electrophysiology systems and methods for guiding intraoperative placement of spinal cord stimulation (SCS) electrode leads and for generating muscle activation maps to facilitate personalized neurostimulation therapy.

### BACKGROUND

Stroke is a leading cause of disability globally, leaving about 400,000 individuals in the United States each year without full use of one or more extremities. Persistent disabilities include hemiplegia, weakness, balance problems, pain, and increased muscle spasticity. People with chronic post-stroke hemiplegia have historically had limited therapeutic options after the subacute rehabilitation period.

### SUMMARY

In an aspect, the disclosure provides a method of adjusting a placement of an electrode of a spinal cord stimulation device in a subject. The method comprises implanting the electrode proximate to a dorsal root of a spinal cord. A series of electrical stimulation pulses are delivered to the electrode. Evoked compound muscle action potentials (CMAP) may be recorded from a plurality of muscles of the subject. The recorded evoked CMAPs may be processed to generate a muscle map associating the electrode with corresponding muscle activation data. An indication for adjusting the placement of the electrode may be generated based on the muscle map. The placement of the electrode may be adjusted according to the indication.

In an aspect, the disclosure provides a method of building a recruitment curve for a spinal cord stimulation electrode array. The method comprises delivering, at a first stimulation frequency of between about 1 Hz and 2 Hz, a series of electrical stimulation pulses to a selected electrode of an electrode array, beginning at or below a motor threshold amplitude. The stimulation amplitude may be incrementally increased until a saturation amplitude is reached for at least one muscle of interest or until a predetermined maximum stimulation amplitude is reached. The evoked CMAP activity may be recorded from a plurality of muscles for each stimulation amplitude. The delivering, incrementally increasing, and recording steps may be recorded for each electrode in the electrode array. A maximum CMAP amplitude may be identified across all electrodes and all stimulation amplitudes. Each individual CMAP amplitude may be normalized for each muscle using the identified maximum CMAP amplitude. A recruitment curve plot of normalized CMAP amplitude versus stimulation amplitude may be generated for each electrode and each muscle.

In an aspect, the disclosure provides a method of automated stimulation parameter selection for spinal cord stimulation therapy in a patient having post-stroke upper limb motor impairment. The method comprises generating a muscle map associating a plurality of implanted spinal cord stimulation electrodes with corresponding muscles of an affected upper limb. A desired motor outcome input may be received from a user. The muscle map and the desired motor outcome may provide input to a recommendation engine. A suggested stimulation configuration, may be received from the recommendation engine, comprising at least a stimulation electrode selection, a stimulation amplitude, a pulse width, and a stimulation frequency.

Further aspects and embodiments are provided in the foregoing drawings, detailed description and claims.

### BRIEF DESCRIPTION OF THE DRAWINGS

The following drawings are provided to illustrate certain embodiments described herein. The drawings are merely illustrative and are not intended to limit the scope of claimed inventions and are not intended to show every potential feature or embodiment of the claimed inventions. The drawings are not necessarily drawn to scale; in some instances, certain elements of the drawing may be enlarged with respect to other elements of the drawing for purposes of illustration.

FIG. 1 is a schematic diagram illustrating an electrophysiology system according to a first embodiment of the invention.

FIG. 2 is a schematic diagram illustrating an electrophysiology system according to a second embodiment of the invention.

FIG. 3 is a set of graphs illustrating frequency-dependent suppression data from an exemplary clinical study (SCS01 and SCS02), showing normalized steady-state CMAP amplitude as a function of stimulation frequency for four representative electrode contacts (7R at 1.7 mA; 2R at 3.3 mA; 1C at 3.0 mA; 7C at 4.2 mA) and seven muscles (A Del, Bic, Pron, Tri, Flx, Ext, ABP), demonstrating that CMAP amplitude decreases with increasing stimulation frequency for contacts exhibiting transsynaptic dorsal root activation.

FIG. 4 is an EMG time-series trace illustrating frequency-dependent suppression from SCS02, Pronator muscle, electrode contact 1C at 3.0 mA, showing high-amplitude consistent CMAPs at 5 Hz, reduced steady-state amplitude at 10 Hz, and further reduced amplitude at 20 Hz, with stimulation markers indicated above the trace (scale bar: 100 μV, 200 ms).

FIG. 5 is a set of EMG traces illustrating stimulus-to-CMAP latency analysis, showing the measurement of latency as the time between the stimulation time marker and the onset of the evoked CMAP (top panel), and comparison between trans-synaptic recruitment characterized by a latency of about 3 ms (bottom left) and direct recruitment characterized by a latency of less than about 1 ms (bottom right), with a scale bar of 100 μV and 200 ms.

FIG. 6 is a set of recruitment curve plots and corresponding anatomical muscle activation maps for three representative electrodes (5R, 2R, 8R), showing normalized peak-to-peak CMAP amplitude versus stimulation current (mA) for each of eleven muscles (TRAP, A DEL, M DEL, P DEL, BIC, TRI, EXT, FLX, PRO, ABP, ADM), with vertical dashed lines indicating representative programming amplitudes (1.4 mA, 1.7 mA, and 1.8 mA respectively), accompanied by anterior and posterior anatomical figures showing color-coded muscle activation maps (normalized 0-1) at the indicated amplitude for each electrode.

FIG. 7 is an anatomical illustration of the posterior cervical spine and torso showing two parallel SCS electrode leads implanted in the cervical epidural space, with the electrode contact array spanning from the upper cervical to thoracic spinal segments.

FIG. 8 is a flow diagram of a method for electrophysiology-guided SCS electrode lead placement.

FIG. 9 is a flow diagram of a method for automated generation of a normalized recruitment curve for a plurality of electrodes.

FIG. 10 is a flow diagram of a method for electrophysiology-guided placement of a multi-electrode SCS array.

FIG. 11 is a flow diagram of a method for computing and plotting a normalized recruitment curve for a single electrode.

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “sensory neuron” refers to a nerve cell of the peripheral nervous system that converts a stimulus into an electrical signal and transmits that signal toward the central nervous system. Sensory neurons are also referred to as afferent neurons. In the context of spinal cord stimulation as described herein, sensory neurons include the primary afferent fibers whose cell bodies are located in the dorsal root ganglia and whose peripheral processes project to sensory receptors in the body while their central processes project into the spinal cord via the dorsal roots. Upon activation by electrical stimulation of the dorsal root or dorsal root entry zone, sensory neurons transmit action potentials centrally through intraspinal synaptic connections to spinal motor neurons, thereby facilitating voluntary motor output via transsynaptic recruitment. Sensory neurons, as used herein, are distinguished from motor neurons, which transmit signals from the central nervous system to effector muscles via the ventral roots.

As used herein, “spinal motor neuron” refers to a lower motor neuron whose cell body is located in the anterior (ventral) horn of the spinal cord gray matter and whose axon projects peripherally through the ventral root to innervate skeletal muscle fibers at the neuromuscular junction, thereby causing muscle contraction. Spinal motor neurons are also referred to as efferent neurons, lower motor neurons, or anterior horn cells. In the context of spinal cord stimulation as described herein, spinal motor neurons may be recruited by two distinct pathways: (1) transsynaptic recruitment, in which electrical stimulation activates sensory afferents in the dorsal root, which in turn excite spinal motor neurons via intraspinal synaptic connections, thereby facilitating voluntary movement without directly depolarizing the motor neuron; and (2) direct recruitment, in which stimulation current directly depolarizes the spinal motor neuron or its axon, bypassing intraspinal synaptic circuitry and producing uncontrolled muscle contraction independent of cortical intent. As used herein, the terms “alpha motor neuron” and “spinal motor neuron” may be used interchangeably to refer to the large-diameter somatic motor neurons that directly innervate extrafusal skeletal muscle fibers and constitute the primary cellular substrate of voluntary movement.

As used herein, “spinal cord stimulation electrode” or “SCS electrode” refers to an electrically conductive contact configured to deliver electrical stimulation energy to neural tissue proximate to the spinal cord when implanted or positioned within the epidural space, intradural space, or other spinal canal compartment. An SCS electrode may be a discrete conductive element (also referred to as a “contact” or “electrode contact”) forming one of a plurality of electrically isolated conductive elements arranged along a spinal cord stimulation lead (also referred to as an “SCS lead” or “electrode lead”). The SCS lead comprises an elongate, flexible, implantable body housing one or more insulated conductors electrically connecting each electrode contact to a proximal connector adapted for coupling to a pulse generator. SCS electrodes and leads may take the form of a percutaneous cylindrical lead, comprising a plurality of ring-shaped electrode contacts circumferentially disposed about the lead body, or a paddle lead (also referred to as a “surgical lead”), comprising a plurality of electrode contacts arranged in one or more columns on a flat, paddle-shaped substrate. As used herein, the term “SCS electrode” encompasses both individual electrode contacts and multi-contact electrode arrays and includes both externalized configurations in which the lead is implanted but the proximal connector is temporarily accessible outside the body during an intraoperative procedure, and fully implanted configurations in which the lead is connected to a subcutaneously implanted pulse generator.

As used herein, “motor threshold” refers to the minimum stimulation intensity required to reliably evoke a measurable compound muscle action potential (CMAP) in at least one muscle of interest in response to electrical stimulation delivered to a spinal cord stimulation (SCS) electrode. The motor threshold is expressed as a current amplitude in milliamperes (mA) and may be determined by delivering stimulation pulses at a low frequency of less than about 2 Hz and incrementally increasing the stimulation amplitude from a sub-threshold level until a CMAP of predefined minimum peak-to-peak amplitude may be consistently detected in at least one recorded muscle. As used herein, “reliably evoke” means that a CMAP response is detected on each of a plurality of successive stimulation pulses at the threshold amplitude. The motor threshold may be an indicator of the proximity of the stimulating electrode to excitable neural structures: a low motor threshold (e.g., less than about 3 mA under general anesthesia) suggests that the electrode may be in close proximity to a dorsal root or its entry zone; a high motor threshold (e.g., greater than about 5 mA under general anesthesia) suggests that the electrode may be farther from the target neural structures, such as when positioned too medially or between dorsal roots on the rostro-caudal axis be. Motor threshold values may vary between individuals and between the anesthetized and awake states; accordingly, motor threshold values as used herein, unless otherwise stated, refer to values obtained intraoperatively under general anesthesia.

As used herein, the term “muscle action potential” refers to the transient electrical depolarization and repolarization of the membrane of a skeletal muscle fiber that is triggered by activation of the neuromuscular junction. A muscle action potential is generated when a motor neuron releases acetylcholine at the neuromuscular junction, causing depolarization of the muscle fiber membrane that, upon reaching threshold, initiates a self-propagating action potential along the length of the fiber. The muscle action potential is an all-or-nothing event: a given muscle fiber either generates a full-amplitude action potential or none at all, depending on whether the membrane potential reaches threshold. A muscle action potential has a duration of about 2-10 milliseconds and propagates bidirectionally along the muscle fiber from the endplate toward the tendinous ends at a conduction velocity of about 2-5 meters per second. The electrical signal associated with a muscle action potential may be detected extracellularly by a surface or intramuscular electrode placed in proximity to the muscle fiber.

As used herein, the term “compound muscle action potential” or “CMAP” refers to the summated electrical signal recorded from a muscle by a surface or intramuscular electrode, representing the near-simultaneous superposition of individual muscle action potentials from a population of muscle fibers belonging to one or more motor units activated by a common neural stimulus. A CMAP may also be referred to in the art as a “compound motor action potential” or “M-wave.” A CMAP may be characterized by its peak-to-peak amplitude (expressed in microvolts, μV, or millivolts, mV), its duration (expressed in milliseconds), its latency (defined as the time from the onset of the evoking stimulus to the onset of the CMAP waveform, expressed in milliseconds), and its waveform morphology. The amplitude of a CMAP may be proportional to the number of muscle fibers activated and may be used herein as a measure of the degree of motor unit recruitment produced by spinal cord stimulation. As used herein, the term “evoked CMAP” refers to a CMAP recorded in response to a discrete electrical stimulation pulse delivered to a spinal cord stimulation electrode, as distinguished from spontaneous or voluntary muscle activity. An evoked CMAP is time-locked to the stimulation pulse and may be identifiable in the electromyography (EMG) recording by its consistent latency and waveform shape relative to the stimulation event. The peak-to-peak amplitude of an evoked CMAP, measured as the voltage difference between the maximum positive and maximum negative deflections of the waveform, may be a metric used in the systems and methods described herein for motor threshold determination, frequency-dependent suppression analysis, and recruitment curve generation.

Epidural cervical spinal cord stimulation (SCS) has been demonstrated to effectively restore motor control of muscles in the corresponding myotome of the spinal segment being activated. Stimulation of the cervical spinal cord spanning spinal segments C3-T1 has been shown to restore upper limb movement in hemiplegic patients following a stroke, including patients assessed years following the stroke event. Unlike functional electrical stimulation (FES), which directly stimulates muscles, SCS stimulates sensory neurons projecting to spinal motor neurons and networks regulating motor output. As a result, SCS can restore volitional movement, and does so only when the patient intentionally attempts to move. SCS is also anatomically specific: only muscles innervated by the spinal segments being activated are affected, allowing personalized, targeted therapy.

Effective SCS for motor function restoration requires precise placement of electrodes (leads) over the dorsal root entry zone of the cervical spinal cord. Placement errors in the medial-lateral or rostral-caudal directions result in degraded therapeutic outcomes. If the electrodes are placed too lateral the motor neurons may be activated. The dorsal and ventral roots converge near the lateral aspect of the spinal cord, increasing the risk of co-activation of ventral motor roots. Direct activation of ventral motor neurons does not leverage the transsynaptic mechanisms needed for volitional movement restoration and may result in uncontrolled direct muscle activation.

If the electrodes are placed too medial the contralateral side may be affected. Electrodes positioned too medially are farther from the dorsal root structures and require higher current amplitudes to activate targeted cells. Stimulation becomes less selective, and there may be an increased risk of crossover effects on the contralateral side of the spinal cord—an especially significant concern in hemiplegic stroke patients whose unaffected limb retains full sensory function.

Due to the rostro-caudal organization of the cervical spinal cord, proximal upper limb muscles (e.g., trapezius, deltoid, biceps) are innervated by rostral segments (C3-C6), while distal muscles (e.g., wrist flexors/extensors, intrinsic hand muscles) are innervated by caudal segments (C7-T1). Insufficient rostro-caudal lead coverage prevents activation of the full complement of targeted muscles.

Leads placed too laterally may slip between dorsal roots and migrate toward the ventral side of the spinal cord, dramatically increasing direct motor neuron activation and rendering the therapy ineffective.

Intraoperative fluoroscopy may be used to guide SCS lead placement, but imaging alone lacks the resolution to reliably confirm electrode placement relative to the dorsal root entry zone. Conventional intraoperative neurophysiology monitoring (IOM) systems are capable of capturing stimulation-triggered EMG, but they rely on manual configuration of each stimulation pattern, cannot interpret the acquired electrophysiology data, and have no standardized means to connect to SCS leads or to automatically analyze data in real time.

Following successful lead placement, programming SCS for motor function restoration requires building a “muscle map” which is a dataset relating each implanted electrode to the muscles it activates and the stimulation amplitudes required for activation. This process, performed by incrementally stepping through stimulation parameters electrode-by-electrode while recording EMG from multiple muscles, is time-consuming, error-prone, and not feasible with existing commercial SCS systems or IOM devices.

The disclosure provides an automated electrophysiology system and associated methods for guiding intraoperative placement of cervical SCS electrode leads and for generating personalized muscle activation maps used during SCS therapy programming. The electrophysiology system is designed for use in restoration of upper limb motor function in patients with chronic hemiplegia resulting from stroke, traumatic brain injury, spinal cord injury, cerebral palsy, or other corticospinal tract disorders, although its use is not limited to these indications.

The electrophysiology system comprises hardware and software components that work in concert to: (1) automatically administer standardized stimulation protocols to SCS electrodes while recording evoked EMG from multiple upper limb muscles; (2) automatically analyze the resulting electrophysiology data to generate placement guidance; and (3) build, visualize, and store muscle activation maps for downstream programming.

The cervical spinal cord, spanning segments C3-T1, innervates the upper extremity musculature in a rostro-caudal somatotopic arrangement. The rostral segments (C3-C4) innervate proximal muscles such as the trapezius and anterior, medial, and posterior deltoid. Intermediate segments (C5-C6) innervate the biceps and forearm musculature, and caudal segments (C7-T1) innervate the wrist flexors, wrist extensors, and intrinsic hand muscles (e.g., abductor pollicis, abductor digiti minimi).

Epidural SCS delivered over the dorsal root entry zone activates sensory neurons in the dorsal roots. These neurons synaptically excite spinal motor neurons, biasing them toward threshold and facilitating volitional movement by enhancing the efficacy of descending corticospinal input. This transsynaptic mechanism may be distinct from direct motor neuron activation achieved by ventral root stimulation; the latter produces uncontrolled direct muscle contraction rather than facilitated voluntary movement.

The electrophysiology system may utilize one or all of three primary electrophysiological biomarkers to assess lead placement relative to the dorsal root entry zone: (1) motor threshold, (2) frequency-dependent suppression, and (3) stimulation latency. Additionally, muscle activation mapping-both ipsilateral and contralateral-provides rostro-caudal coverage confirmation and detects inadvertent crossover activation.

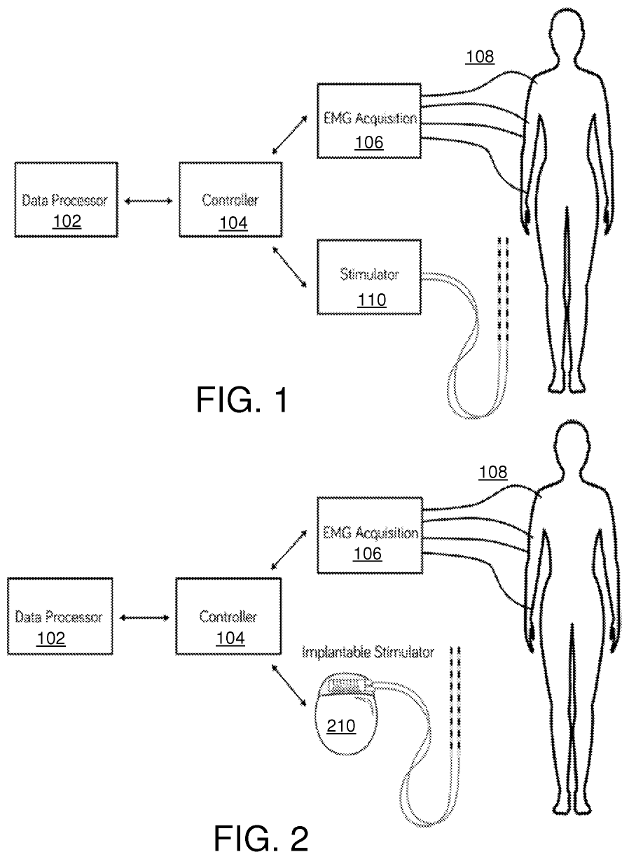

Referring to FIG. 1, an electrophysiology system 100 according to a first embodiment comprises a data processor 102, a controller 104, an EMG acquisition subsystem 106, a plurality of EMG electrodes 108 placed on the surface of or inserted into muscles of a subject, and a stimulator 110 connected to the externalized proximal end of one or more implanted SCS electrode leads. The controller 104 communicates bidirectionally with the data processor 102. The controller 104 further communicates with the EMG acquisition subsystem 106 to trigger data acquisition and with the stimulator 110 to command delivery of electrical stimulation pulses with specified parameters including electrode selection, current amplitude, pulse width, and frequency. The EMG acquisition subsystem 106 records differential EMG signals from the EMG electrodes 108 and communicates the time-series data to the controller 104 and data processor 102.

In the embodiment of FIG. 1, the stimulator 110 may be an external stimulation device capable of reversibly connecting to the externalized leads of an implanted SCS electrode array intraoperatively, prior to surgical closing. This configuration may be used for intraoperative use during the lead implantation procedure.

Referring to FIG. 2, an electrophysiology system 200 according to a second embodiment similar to electrophysiology system 100 except that the stimulator 110 may be replaced by an implantable stimulator 210. In this embodiment, the implantable stimulator 210 has already been fully implanted and may be used as the source of spinal cord stimulation, while the data processor 102, controller 104, EMG acquisition subsystem 106, and EMG electrodes 108 remain as external components. This configuration may be suited for post-implant programming sessions and for ongoing optimization of stimulation parameters. The implantable stimulator 210 communicates with the controller 104 via a wireless communication link (e.g., Bluetooth Low Energy, near-field telemetry, or equivalent).

In both embodiments, the controller 104 may be configured to time-synchronize stimulation pulse delivery with EMG data acquisition so that the precise timing relationship between each stimulation event and the resulting CMAP response is known. Synchronization may be achieved by: (a) a shared hardware clock signal between the stimulator and the EMG acquisition subsystem; (b) an in-line current sensor interposed between the stimulator output and the electrode lead connection, whose output may be recorded on a dedicated channel of the EMG acquisition subsystem; or (c) recording stimulation artifact on the EMG acquisition channels and using artifact onset timing to reconstruct the stimulation timestamp.

In embodiments, the stimulator may be configured to deliver monophasic or biphasic, cathodic-first or anodic-first, constant-current or constant-voltage electrical stimulation pulses to any of a plurality of SCS electrodes (e.g., up to 16 channels). Stimulation parameters may be software-configurable and may include frequency, amplitude, pulse width, and electrode configuration. In embodiments, the frequency may be from about 0.1 Hz to about 10,000 Hz, or from about 0.5 Hz to about 60 Hz for intraoperative use. In embodiments, the frequency may be about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, or 60 hz.

In embodiments, the amplitude of the stimulation may be about 0 mA to about 20 mA in steps of about 0.1 mA (constant-current mode); or corresponding voltage range in constant-voltage mode. In embodiments, the amplitude may be about 0.0, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, or 20.0 mA.

In embodiments, the pulse width may be from about 100 μs to about 1000 μs. In some embodiments, the pulse width may be from about 100 μs to about 500 μs. In some embodiments, the pulse width may be about 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500 μs.

In embodiments the electrode may be configured as monopolar (single active contact with remote return). In embodiments, the electrode may be configured as multipolar (multiple active and/or return contacts)

In some embodiments, the stimulator is capable of wireless communication, for example via Bluetooth Low Energy (BLE), with a control application executing on a connected computing device.

In certain embodiments, the stimulator may be an external pulse generator (EPG) that can be reversibly connected to externalized SCS leads intraoperatively.

In some embodiments, an in-line current sensor may be interposed between the stimulation driver and the SCS lead connection. The current sensor comprises precision low-value sense resistors—one per stimulation channel—that convert stimulation current to a proportional voltage. This voltage signal may be recorded by the EMG acquisition subsystem, providing the acquisition subsystem with precise timing, amplitude, and electrode identification information for each stimulation pulse. This enables accurate stimulus-triggered EMG capture without requiring the stimulation driver to provide a separate synchronization signal to the acquisition hardware, thereby allowing the electrophysiology system to operate with any compatible stimulation driver.

In alternative embodiments, synchronization between the stimulation driver and EMG acquisition subsystem may be achieved by: (a) sharing a hardware clock signal between the two subsystems; (b) recording stimulation artifact on the EMG acquisition channels and using the artifact timing to reconstruct stimulation event timestamps; or (c) software-based timestamp alignment using a shared network time protocol (NTP) reference.

The acquisition subsystem may be configured to capture “triggered” EMG epochs time-locked to each stimulation pulse, enabling precise measurement of stimulus-to-response latency and CMAP characteristics.

The controller 104 may be implemented as a software application executing on a personal computer (PC) or equivalent computing platform. It may be responsible for: specifying and commanding stimulation parameters to the stimulator 110 or 210; triggering EMG data acquisition from the EMG acquisition subsystem 106; time-stamping stimulation events; and routing acquired time-series data to the data processor 102.

The data processor 102 receives time-series EMG data with associated stimulation metadata (electrode index, amplitude, pulse width, frequency, and event timestamp), and may perform the following automated analyses: CMAP detection and characterization; motor threshold determination; frequency-dependent suppression analysis; stimulation latency analysis; ipsilateral and contralateral muscle activation mapping; and recruitment curve generation. The data processor 102 generates placement guidance outputs and muscle map outputs and may push results to a cloud-based data management platform for storage and downstream use.

The EMG acquisition subsystem 106 may simultaneously record differential EMG signals from a plurality of muscle channels. In certain embodiments, EMG may be recorded from at least the following muscles of the affected upper limb: trapezius (TRAP; C3-C4), anterior deltoid (A DEL; C4-C5), medial deltoid (M DEL; C4-C5), posterior deltoid (P DEL; C4-C5), biceps brachii (BIC; C5-C6), triceps brachii (TRI; C6-C8), wrist flexor (FLX; C6-C7), wrist extensor (EXT; C7-C8), forearm pronator (PRO; C6-C7), forearm supinator (C5-C6), abductor pollicis (ABP; C8-T1), and abductor digiti minimi (ADM; C8-T1). EMG may additionally be recorded from at least biceps brachii (C4-C6) and wrist extensor (C7-C8) of the unaffected contralateral limb, to enable detection of inadvertent crossover stimulation.

The EMG acquisition subsystem 106 may accept intramuscular needle electrodes or surface electrodes via standard medical connectors, may sample at a rate of from about 250 samples per second to about 10,000 samples per second per channel, and may provide a common-mode rejection ratio (CMRR) of from about 20 dB to about 100 dB and an input impedance of from 0.5 MΩ to about 100 MΩ.

In some embodiments, the electrophysiology system may optionally include a cloud-based data management platform (e.g., implemented on Amazon Web Services or equivalent). The cloud based management platform may: store muscle map data, raw electrophysiology time-series, patient stimulation history, device configurations, and clinician/patient feedback; perform serverless pre-processing of incoming electrophysiology data; support aggregation and anonymization of outcomes data across patients for large-scale analytics and algorithm development; provide bidirectional data communication with the electrophysiology PC application, the programming interface, and the implantable pulse generator programmer; and comply with applicable data privacy regulations (e.g., HIPAA) and cybersecurity standards.

In certain embodiments, the electrophysiology system may optionally include a programming user interface. The programming user interface may be implemented as a mobile application on a tablet (e.g., iPad running iOS or an Android tablet). In embodiments, the programming user interface may display a muscle activation map showing the relative percent-activation of each upper limb muscle for a given stimulation configuration. In some embodiments, the programming user interface may enable outcome-centric stimulation adjustment. For example, a clinician or subject may adjust sliders corresponding to desired muscle activations, and the interface may translate these high-level requests into specific stimulation parameter changes using the muscle selectivity index algorithm. In certain embodiments, the programming user interface may integrate an LLM-based recommendation engine that accepts natural language input describing a desired motor outcome and returns recommended stimulation parameter adjustments and may use patient-specific history for context-primed personalization.

### Motor Threshold Analysis

The data processor 102 may automatically determine the motor threshold for each stimulation electrode, defined as the minimum stimulation current amplitude sufficient to elicit an identifiable CMAP in at least one target muscle. To determine the motor threshold, the data processor 102 initiates stimulation at a frequency of less than about 2 Hz, beginning at 0 mA. The data processor 102 then incrementally increases the stimulation amplitude in steps of about 0.1 mA, waiting for a sufficient number of stimulation pulses at each amplitude to reliably detect a CMAP response. In some embodiments, a binary search algorithm may be employed in place of a linear amplitude sweep to converge on the motor threshold more rapidly. The motor threshold may be identified as the lowest stimulation amplitude at which a CMAP of a predefined minimum peak-to-peak amplitude may be consistently observed in at least one recorded muscle.

Based on the determined motor threshold, the data processor 102 may interpret the result and may report a placement guidance output to the user. A motor threshold of less than about 3 mA suggests that the stimulating electrode may be in close proximity to the dorsal root entry zone; however, a threshold in this range may also indicate direct ventral root activation, and supplemental analysis using frequency-dependent suppression or latency measurements is recommended to distinguish between these two conditions. A motor threshold of about 3-5 mA may represent acceptable electrode placement but warrants further analysis to confirm. A motor threshold of greater than about 5 mA indicates that the electrode may be farther from the target dorsal root structures than desired, which may result from overly medial positioning or from the electrode being situated between dorsal roots on the rostro-caudal axis; in such cases, the data processor 102 recommends lateral adjustment of the lead or evaluation of adjacent electrode contacts. It should be noted that motor threshold values may differ between the anesthetized and awake states; unless otherwise stated, motor threshold values referenced herein reflect intraoperative values obtained under general anesthesia.

### Frequency-Dependent Suppression Analysis

Transsynaptic activation of spinal motor neurons via sensory neurons exhibits a characteristic phenomenon known as frequency-dependent suppression. At low stimulation frequencies of less than about 2 Hz, each stimulation pulse evokes a consistent, high-amplitude CMAP, as the sensory neuron-to-motor neuron synaptic connection has sufficient time to fully recover between pulses. At higher stimulation frequencies, however, synaptic fatigue causes the CMAP amplitude to progressively decline from an initial transient response to a reduced steady-state value, because a portion of the neurons in the circuit are unable to fully recover between successive pulses. Direct activation of motor neurons via ventral root stimulation does not exhibit this characteristic amplitude suppression at the same frequencies, because the response depends on the axonal firing rate of the motor neuron rather than on the recovery kinetics of a synaptic connection. The data processor 102 exploits this distinction to automatically detect whether the evoked CMAPs are produced by transsynaptic sensory neuron activation or by direct motor neuron activation.

The data processor 102 may automatically detect frequency-dependent suppression using one of two protocols. In a first protocol (Protocol A, high-frequency single recording), stimulation may be delivered at or above the motor threshold at a frequency of at least about 20 Hz in some embodiments, and from about 40 Hz to about 60 Hz in certain embodiments. The data processor 102 analyzes the resulting CMAP time-series for a transient initial high-amplitude response that decays to a lower steady-state amplitude over the course of several successive stimulation pulses. If such a transient-to-steady-state decay may be observed, frequency-dependent suppression may be confirmed, indicating that the evoked CMAPs are produced by transsynaptic activation of spinal motor neurons via sensory afferents in the dorsal root. In a second protocol (Protocol B, stepped frequency), stimulation may be first delivered at or above the motor threshold at a frequency of less than about 2 Hz, and the steady-state CMAP amplitude may be recorded. The stimulation frequency may then be increased in increments of about 10 Hz, with the CMAP amplitude allowed to reach steady state at each frequency before the steady-state value may be recorded. This process may be repeated up to at least about 20-40 Hz. Frequency-dependent suppression may be confirmed when the recorded steady-state CMAP amplitude decreases monotonically with each incremental increase in stimulation frequency. If frequency-dependent suppression is not observed under either protocol, the data processor 102 alerts the user that the electrode contacts may be positioned too laterally, near the zone of convergence of the dorsal and ventral roots, or that the lead may have migrated ventrally around the lateral aspect of the spinal cord. In either case, the data processor 102 recommends corrective adjustment of the lead placement.

### Stimulation Latency Analysis

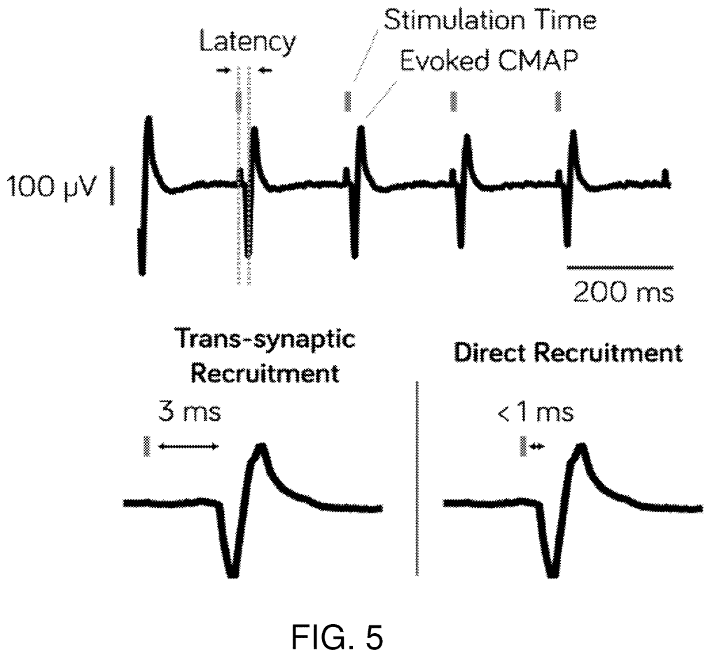

The stimulus-to-CMAP latency may provide an additional electrophysiological biomarker that the data processor 102 uses to distinguish direct motor neuron activation from transsynaptic activation via sensory afferents. As described above with reference to FIG. 5, synaptic transmission across a single synapse adds about 2-3 ms to the response latency relative to the latency expected from direct axonal conduction alone. Accordingly, the latency between a stimulation pulse and the onset of the resulting evoked CMAP reflects whether one or more synaptic connections are interposed between the stimulated neural structure and the activated motor neurons.

To perform stimulation latency analysis, the data processor 102 directs delivery of stimulation pulses at a frequency of less than about 2 Hz on a selected electrode contact. For each stimulation pulse that produces a detectable evoked CMAP, the data processor 102 measures the duration from the onset of the stimulation pulse to the onset of the resulting CMAP, defined as the time point at which the CMAP waveform first exceeds a predefined threshold amplitude above the baseline noise floor. The data processor 102 then interprets the measured latency to classify the mode of motor neuron recruitment. A latency below a predefined minimum value consistent with direct axonal conduction indicating that insufficient time has elapsed for synaptic transmission to have occurred may be classified as direct motor neuron activation, and the data processor 102 may generate a placement guidance output recommending corrective adjustment of the lead. A latency consistent with at least one synaptic delay, defined as a latency of greater than about 3 ms above the expected direct conduction latency, is classified as indirect transsynaptic activation, indicating that the stimulation is recruiting motor neurons via one or more intervening synaptic connections rather than by direct depolarization of the motor neuron or its axon. In such cases, the data processor 102 reports that transsynaptic activation is occurring and recommends that supplemental frequency-dependent suppression analysis be performed to further confirm that the transsynaptic pathway involves sensory afferents in the dorsal root, as opposed to other indirect pathways.

### Muscle Activation Mapping Ipsilateral (Rostro-Caudal Placement)

The data processor 102 may automatically perform rostral and caudal placement confirmation by evaluating which muscles are activated by the most rostral and most caudal electrode contacts of the implanted array. To confirm rostral placement, the data processor 102 directs delivery of stimulation pulses to the most rostral electrode contact at a frequency of less than about 2 Hz, incrementally increasing the stimulation amplitude until the motor threshold is reached. The data processor 102 then evaluates whether a CMAP is evoked in the trapezius muscle, which is innervated by spinal segment C3-C4 and serves as the primary indicator of adequate rostral coverage. If a CMAP is detected in the trapezius, rostral coverage to the C3-C4 level is confirmed and correct rostral positioning is indicated. If CMAPs are detected only in more distally innervated muscles, such as the deltoid or biceps, but not in the trapezius, the data processor 102 generates a placement guidance output indicating that the electrode array must be advanced further rostrally. If no CMAP is detected at expected stimulation amplitudes in any muscle, the data processor 102 recommends attempting stimulation at a higher amplitude or repositioning the array rostrally.

To confirm caudal placement, the data processor 102 directs delivery of stimulation pulses to the most caudal electrode contact at a frequency of less than about 2 Hz, incrementally increasing the stimulation amplitude to the motor threshold. The data processor 102 evaluates whether CMAPs are evoked in the intrinsic hand muscles, specifically the abductor pollicis and abductor digiti minimi, which are innervated by spinal segment T1 and serve as the primary indicators of adequate caudal coverage. If CMAPs are detected in one or more intrinsic hand muscles, caudal coverage to the T1 level is confirmed and correct caudal positioning may be indicated. If no CMAP is detected in the intrinsic hand muscles, the data processor 102 directs stimulation of the adjacent more rostral electrode contact to determine whether hand muscle CMAPs can be obtained at that contact; if hand muscle activity is evoked by the adjacent rostral contact but not by the most caudal contact, the data processor 102 indicates that the most caudal electrode may be positioned beyond T1 and recommends withdrawing the array slightly in the rostral direction. If no CMAP response is obtained at any electrode contact, the data processor 102 indicates that the electrode array may be only partially inserted into the epidural space and recommends further advancement of the lead.

### Muscle Activation Mapping—Contralateral

In addition to ipsilateral muscle activation mapping, the data processor 102 monitors for inadvertent crossover stimulation to the contralateral unaffected limb throughout the mapping procedure. To enable contralateral monitoring, EMG electrodes are placed on at least two muscles of the unaffected limb prior to the procedure, comprising at minimum the biceps brachii, which may be innervated by spinal segments C4-C6, and the wrist extensor, which is innervated by spinal segments C7-C8, providing coverage of the majority of the clinically relevant cervical spinal segments without requiring a full complement of contralateral EMG electrodes. During execution of the ipsilateral mapping protocols described above, the data processor 102 continuously monitors the contralateral EMG channels for the presence of evoked CMAPs time-locked to the stimulation pulses. If contralateral CMAPs are detected above a predefined amplitude threshold, the data processor 102 generates an alert indicating that the lead may be positioned too medially, such that the stimulation current may be spreading across the spinal midline to activate contralateral spinal structures, or that the lead may have crossed the midline entirely. In either case, the data processor 102 recommends lateral repositioning of the lead on the ipsilateral side to reduce the stimulation spread to contralateral neural structures.

### Recruitment Curve Generation

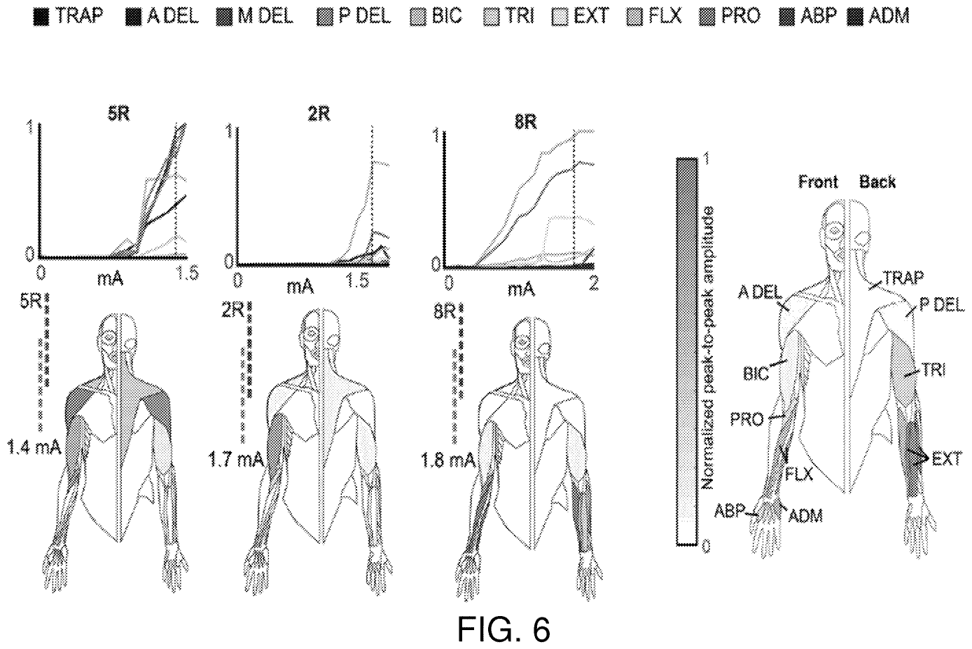

The data processor 102 generates a normalized recruitment curve for each electrode-muscle pair to characterize the stimulation selectivity and dynamic range of each electrode contact. To generate the recruitment curves, the data processor 102 directs delivery of stimulation pulses to a given electrode contact at a frequency of less than about 2 Hz across a range of stimulation amplitudes beginning at or below the motor threshold and increasing to a defined maximum amplitude in some embodiments, about 11-12 mA, or the saturation amplitude of the muscles of interest, whichever is reached first. For each stimulation pulse and each recorded muscle channel, the data processor 102 extracts the peak-to-peak amplitude of the evoked CMAP. This process may be repeated for each electrode contact of interest in the array. Upon completion of the amplitude sweep across all electrodes, the data processor 102 identifies, for each recorded muscle, the maximum CMAP amplitude observed across all electrode contacts and all stimulation amplitudes, which is taken as the maximum obtainable activation of that muscle under the current recording configuration. Each individual CMAP amplitude measurement for a given muscle may be then normalized to that muscle's maximum value, yielding a percent-of-maximum activation value ranging from 0 to 1. The data processor 102 generates a recruitment curve by plotting the normalized CMAP amplitude as a function of stimulation amplitude for each electrode-muscle pair, as illustrated in FIG. 6.

To quantify the specificity of activation for a given target muscle on a given electrode at a given stimulation intensity, the data processor 102 computes a muscle selectivity index (SI). For a target muscle i stimulated by a given electrode at stimulation intensity [symbol] , the muscle selectivity index is defined as:

$${SI}_{i} ( I ) = \frac{{u}_{i}}{{\sum }_{j = 1}^{n} {u}_{j}}$$

where uᵢ is the normalized CMAP activity of target muscle i, and

$${\sum }_{j = 1}^{n}$$

uⱼ is the sum of the normalized CMAP activities across all [symbol] muscles activated by that stimulation configuration. A muscle selectivity index approaching 1.0 for a given target muscle indicates that the electrode activates that muscle with high specificity relative to all other recorded muscles at the tested amplitude, while a value approaching 0 indicates that the electrode activates the target muscle non-selectively relative to the broader muscle population. The data processor 102 identifies, for each recorded muscle, the electrode contact having the highest muscle selectivity index at one or more clinically relevant stimulation amplitudes and reports this electrode as the recommended contact for targeting that muscle during SCS therapy programming.

### Intraoperative Protocol

An exemplary automated intraoperative protocol for guiding lead placement using the electrophysiology system 100 and methods 800 and 1000 is described below. The steps of the protocol may be performed automatically under the direction of the controller 104 and data processor 102, with iterative positional adjustments made by the surgeon based on the placement guidance outputs generated by the electrophysiology system 100.

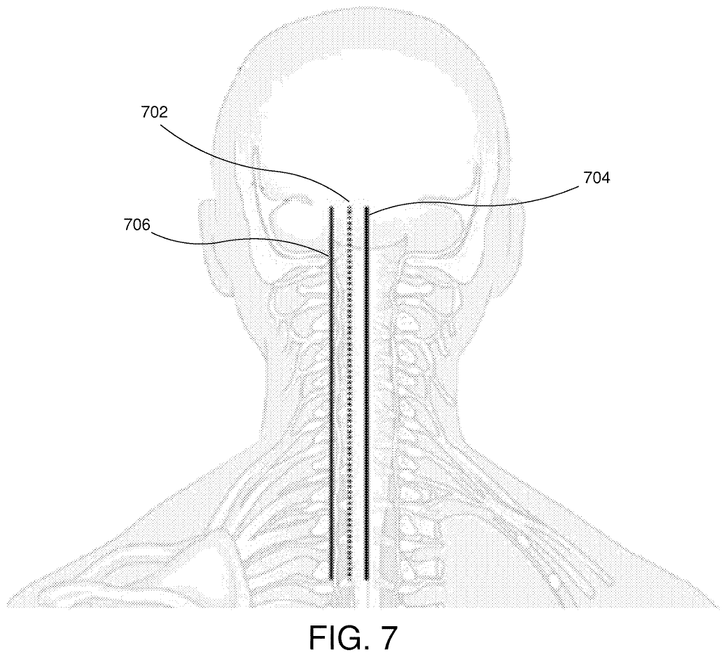

At the outset of the procedure, the surgeon uses intraoperative fluoroscopy to advance the electrode array into the cervical epidural space and position the most rostral electrode contact near the C3-C4 intervertebral interface, as illustrated in FIG. 7. Following initial positioning, the controller 104 directs delivery of stimulation pulses to the most rostral electrode contact at a frequency of less than about 2 Hz with incrementally increasing amplitude, and the data processor 102 evaluates whether CMAPs are evoked in the trapezius muscle, which is innervated by spinal segment C3-C4 and serves as the primary indicator of adequate rostral coverage. If CMAPs are detected only in more distally innervated muscles, such as the deltoid or biceps, but not in the trapezius, the data processor 102 generates a placement guidance output indicating that the array requires further rostral advancement.

The controller 104 then directs stimulation of the most caudal electrode contact at a frequency of less than about 2 Hz with incrementally increasing amplitude, and the data processor 102 evaluates whether CMAPs are evoked in the intrinsic hand muscles, specifically the abductor pollicis (ABP) and abductor digiti minimi (ADM), which are innervated by spinal segment T1 and serve as the primary indicators of adequate caudal coverage. If no hand muscle response is observed, the data processor 102 may recommend stimulation of an adjacent more rostral contact to rule out the possibility that the most caudal contact is positioned beyond T1, in which case the array may need to be withdrawn slightly in the rostral direction.

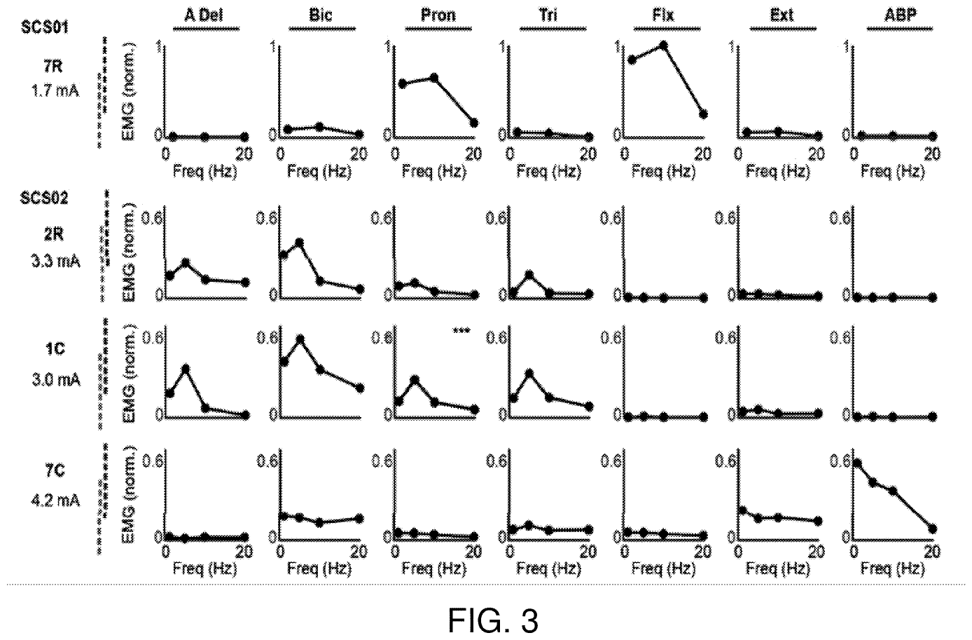

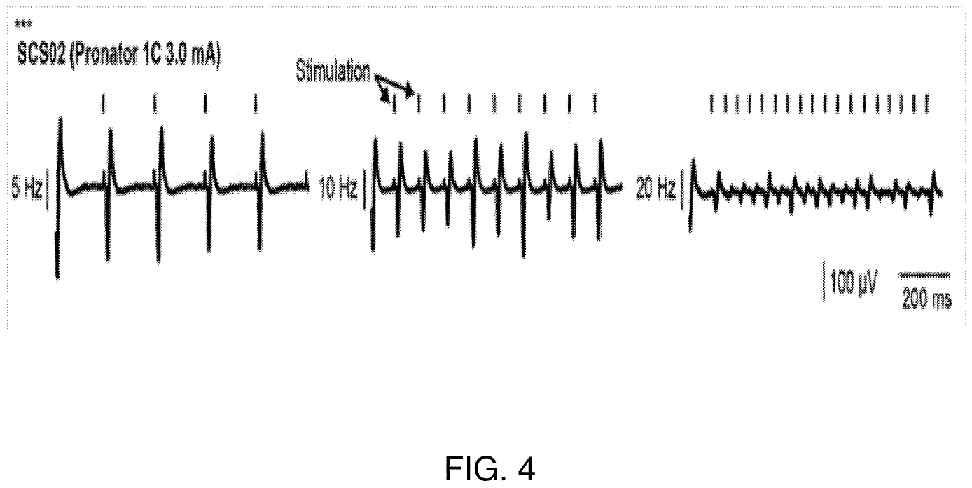

Once satisfactory rostro-caudal coverage is confirmed, the electrophysiology system 100 performs frequency-dependent suppression analysis, as described above with reference to FIGS. 3 and 4, on two to three electrode contacts distributed along the length of the array. The data processor 102 identifies any contacts that exhibit absent frequency-dependent suppression, which may be indicative of direct motor neuron activation via the ventral root rather than transsynaptic activation via dorsal root sensory afferents, and recommends lateral repositioning of the lead to bring those contacts into closer proximity to the dorsal root entry zone. Throughout all steps of the protocol, the data processor 102 continuously monitors the contralateral EMG channels, and if CMAPs are detected on the unaffected limb at any point during the procedure, the data processor 102 issues an alert and recommends lateral repositioning of the lead to reduce stimulation spread to the contralateral spinal structures.

If any repositioning adjustment is required at any stage of the protocol, the surgeon partially withdraws and repositions the lead, and the analysis sequence may be repeated from the rostral and caudal mapping steps, or from the frequency-dependent suppression analysis step, as appropriate to the nature of the adjustment made.

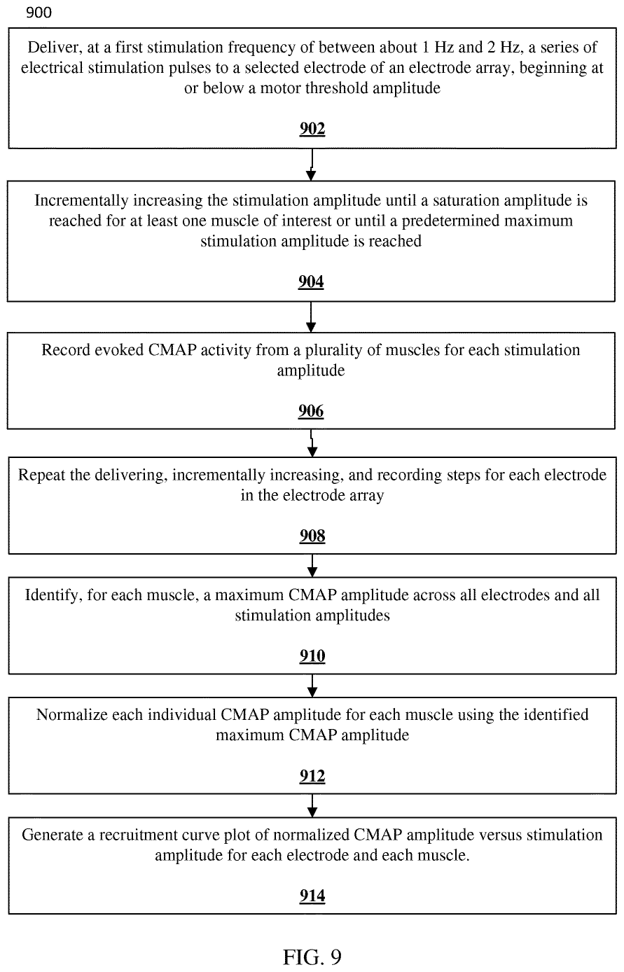

Once satisfactory lead placement has been confirmed across all placement criteria, the data processor 102 automatically executes the full amplitude sweep protocol corresponding to method 900 illustrated in FIG. 9 across all electrode contacts, under the supervision of a trained clinical professional who monitors the procedure and establishes the maximum safe stimulation amplitude for each contact. The data processor 102 then processes the collected electrophysiology data to generate normalized recruitment curves and anatomical muscle activation maps for each electrode contact, as illustrated in FIG. 6. Upon completion of data processing, the electrophysiology system 100 transmits the processed results to the cloud-based data management platform for storage and subsequent use during stimulation programming sessions.

### Stimulation Protocols

The electrophysiology system 100 supports a plurality of pre-programmed stimulation paradigms that are selectable by the user through the controller interface. In a first paradigm, a muscle mapping sweep may be performed by delivering stimulation at about 2 Hz to each electrode contact sequentially, with one, two, or three pulses delivered at each of a series of stimulation amplitudes ranging from 0 mA to about 10 mA, thereby enabling rapid characterization of the motor threshold and muscle activation pattern for each contact across the full array. In a second paradigm, a frequency-dependent suppression assessment may be performed by delivering stimulation at a fixed supra-threshold amplitude at a frequency of between about 20 Hz and 60 Hz on a specified electrode contact, enabling the data processor 102 to evaluate the CMAP time-series for the characteristic transient-to-steady-state amplitude decay indicative of transsynaptic sensory afferent activation. In a third paradigm, a rostro-caudal placement check may be performed by delivering stimulation at about 2 Hz to the most rostral and most caudal electrode contacts at incrementally increasing amplitudes, as described above in Sections IV.D and V. In a fourth paradigm, a manual configuration mode permits the user to select any combination of stimulation parameters across all available settings, providing maximum flexibility for exploratory data collection or for clinical scenarios not covered by the pre-programmed paradigms.

### LLM-Based Recommendation Engine

In embodiments, the electrophysiology system 100 includes an LLM-based recommendation engine configured to assist clinicians and patients during stimulation programming sessions. The recommendation engine may be fine-tuned on a curated corpus of literature relating to neuromodulation, spinal cord stimulation, spinal anatomy, neurophysiology, and clinical programming practice, using Retrieval-Augmented Generation (RAG) techniques to enable the model to access and reason over domain-specific reference material at inference time. The recommendation engine may be further tuned on a dataset of validated prompt-response pairs derived from clinical session recordings, in which patient-described motor outcome goals are paired with the corresponding stimulation parameter adjustments made by experienced clinicians, thereby training the model to translate high-level functional objectives into specific parameter changes. During a programming session, the recommendation engine accepts natural language input from a clinician or patient describing a desired motor outcome for example, a request for increased elbow extension support without reduction in elbow flexion capability and generates a recommended set of stimulation parameter adjustments, including electrode contact selection, stimulation amplitude, and pulse width, accompanied by a natural language explanation of the reasoning underlying the recommendation. To enable personalized responses, a patient-specific history file stored in the cloud-based data management platform may be retrieved and used to prime the model's context prior to inference; this history file contains relevant patient demographics, previous stimulation configurations, the outcomes of prior programming adjustments, and clinician and patient feedback, enabling the recommendation engine to generate responses tailored to the individual patient's neuromuscular profile and therapeutic history. In embodiments, the recommendation engine may be evaluated across multiple foundational large language model architectures including, for example, GPT-series, Claude, Llama, and Gemini models using a standardized set of test prompts and blinded human grader scoring, and the best-performing model architecture may be selected for deployment in the production system.

### Imaging Integration

In some embodiments, the electrophysiology system 100 optionally incorporates intraoperative imaging data, such as fluoroscopic X-ray images, to supplement the electrophysiological placement guidance described above. When present, an imaging analysis module of the data processor 102 processes the intraoperative imaging data to identify the approximate position of each electrode contact relative to vertebral landmarks visible in the fluoroscopic image, enabling correlation of electrode positions with specific cervical and thoracic spinal levels. The imaging analysis module further detects potential lead migration toward the lateral edge of the spinal canal, which would increase the risk of ventral root co-activation, as well as ventral displacement of the lead tip below the dorsal surface of the spinal cord, which can occur when a lead slips between dorsal roots along the lateral aspect of the cord. The imaging analysis module additionally verifies that the lead is positioned within the epidural space and has not been inadvertently placed in an extradural tissue plane, such as the plane between the vertebral lamina and the overlying fascia. Positional estimates derived from imaging analysis are correlated with the electrophysiological biomarkers described above to generate an integrated placement guidance output that draws on both modalities. It is to be understood, however, that not all embodiments of the electrophysiology system 100 include image processing capabilities, and that the electrophysiological analysis components of the electrophysiology system 100 are fully operative as a standalone lead placement guidance system independently of any imaging integration.

### Stimulation Latency as a Supplemental Biomarker

As shown in FIG. 5, stimulus-to-CMAP latency provides an additional biomarker for distinguishing dorsal root (transsynaptic) from ventral root (direct) motor neuron activation. A latency of about 3 ms or greater (consistent with the transit time of at least one synaptic delay) indicates transsynaptic motor neuron recruitment via sensory afferents. A latency of less than about 1 ms indicates that the stimulation current has reached the motor neuron directly, without passing through a synapse. The data processor 102 measures latency for each stimulation pulse and CMAP pair and may use this metric in combination with frequency-dependent suppression and motor threshold analysis to provide a composite placement confidence assessment.

For intraoperative use, paralytic anesthetic agents (e.g., isoflurane) should be discontinued sufficiently in advance of electrophysiological measurements to ensure complete washout, as neuromuscular blocking agents interfere with CMAP generation and may result in artificially elevated motor thresholds or absent CMAP responses. Anesthesia may be maintained using sedative agents (e.g., propofol, sevoflurane) at levels compatible with somatosensory evoked potential monitoring. The electrophysiology system and methods may alternatively be used with awake patients; motor threshold values in awake subjects typically differ from intraoperative values.

The controller 104 need not fully automate the stimulation protocol; it may alternatively provide a user interface through which a clinician manually configures individual stimulation parameters for each test, with the data processor 102 performing automated analysis of the resulting EMG data. The electrophysiology system may optionally include an imaging analysis module configured to receive intraoperative fluoroscopic X-ray data and identify lead position relative to vertebral landmarks, proximity of leads to the lateral edge of the spinal canal, and potential ventral displacement below the dorsal surface of the spinal cord. The electrophysiology system may further include a cloud-connected programming interface, implementing a muscle map-driven outcome-centric stimulation programming workflow and optionally an LLM-based recommendation engine. The methods described herein may be applied to SCS at cervical, thoracic, or lumbar spinal levels and are not limited to upper limb motor function applications.

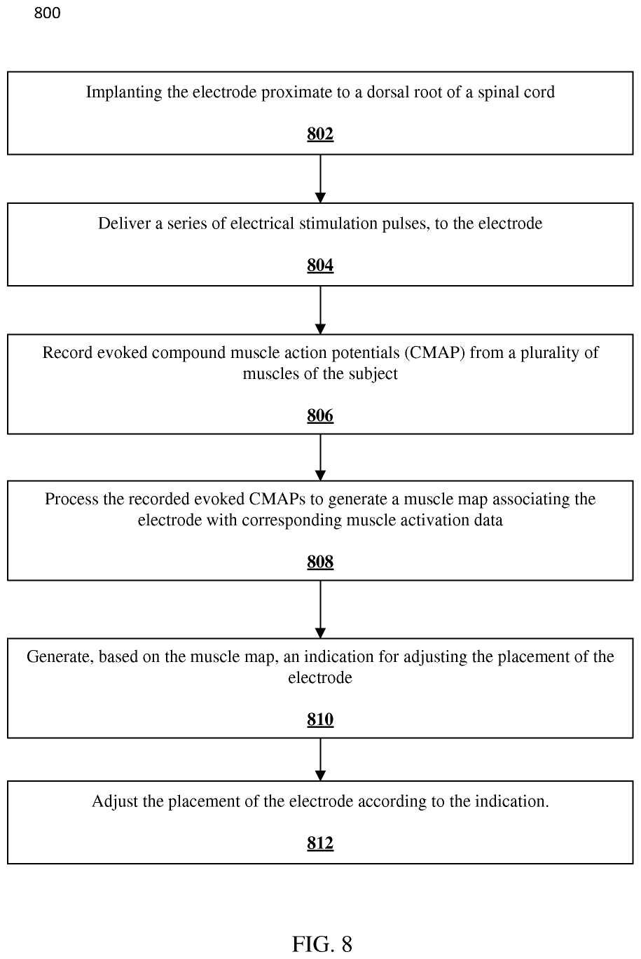

FIG. 8 illustrates a method 800 for electrophysiology-guided SCS electrode placement. At step 802, an electrode is implanted proximate to a dorsal root of the spinal cord of a subject. The electrode may be any of the contacts of a multi-contact SCS lead as shown in FIG. 7. At step 804, a series of electrical stimulation pulses is delivered to the electrode via the stimulator 110 or 210 under control of the controller 104. The stimulation pulses are delivered at a frequency and amplitude selected to elicit detectable CMAPs while minimizing patient discomfort and physiological disruption; in embodiments, a frequency of between about 1 Hz and about 2 Hz is used for initial testing. At step 806, evoked CMAPs are recorded from a plurality of muscles of the subject via the EMG acquisition subsystem 106 and EMG electrodes 108. The recorded CMAPs are time-synchronized with the stimulation pulses via the synchronization interface of the controller 104.

At step 808, the data processor 102 processes the recorded CMAPs to generate a muscle map associating the electrode with corresponding muscle activation data. The muscle map includes, for each muscle channel, the CMAP peak-to-peak amplitude, stimulus-to-CMAP latency, and any frequency-dependent suppression characteristics derived from the recorded data. At step 810, the data processor 102 generates, based on the muscle map, an indication for adjusting the placement of the electrode. The indication may specify one or more of: move electrode rostral (insert further); withdraw electrode caudally; move electrode laterally; move electrode medially; re-confirm placement using supplemental tests; or placement confirmed. At step 812, the placement of the electrode may be adjusted by the surgeon according to the indication. Steps 804-812 may be iteratively repeated until satisfactory placement is achieved.

FIG. 9 illustrates a method 900 for generating normalized recruitment curves for a plurality of electrodes in an electrode array. At step 902, stimulation pulses are delivered to a selected electrode of the electrode array at a first stimulation frequency of between about 1 Hz and 2 Hz, beginning at or below the motor threshold amplitude for that electrode. At step 904, the stimulation amplitude may be incrementally increased until either a saturation amplitude is reached for at least one muscle of interest (i.e., further amplitude increases produce no additional CMAP amplitude increase for that muscle) or until a predetermined maximum stimulation amplitude is reached, whichever occurs first. In embodiments, the maximum stimulation amplitude does not exceed about 11-12 mA; in some embodiments it may be set lower based on clinician judgment. At step 906, evoked CMAP activity from a plurality of muscles is recorded for each stimulation amplitude. At step 908, steps 902-906 are repeated for each electrode in the electrode array. At step 910, for each muscle, a maximum CMAP amplitude may be identified across all electrodes and all stimulation amplitudes tested; this value represents the maximum obtainable activation of that muscle under the current recording configuration and may be used as a normalization reference. At step 912, each individual CMAP amplitude measurement for each muscle may be normalized to the corresponding muscle-specific maximum CMAP amplitude, yielding a percent-of-maximum activation value between 0 and 1. At step 914, a recruitment curve plot of normalized CMAP amplitude (y-axis, 0-1) versus stimulation amplitude in mA (x-axis) may be generated for each electrode-muscle pair and displayed on the user interface, as illustrated in the upper panels of FIG. 6.

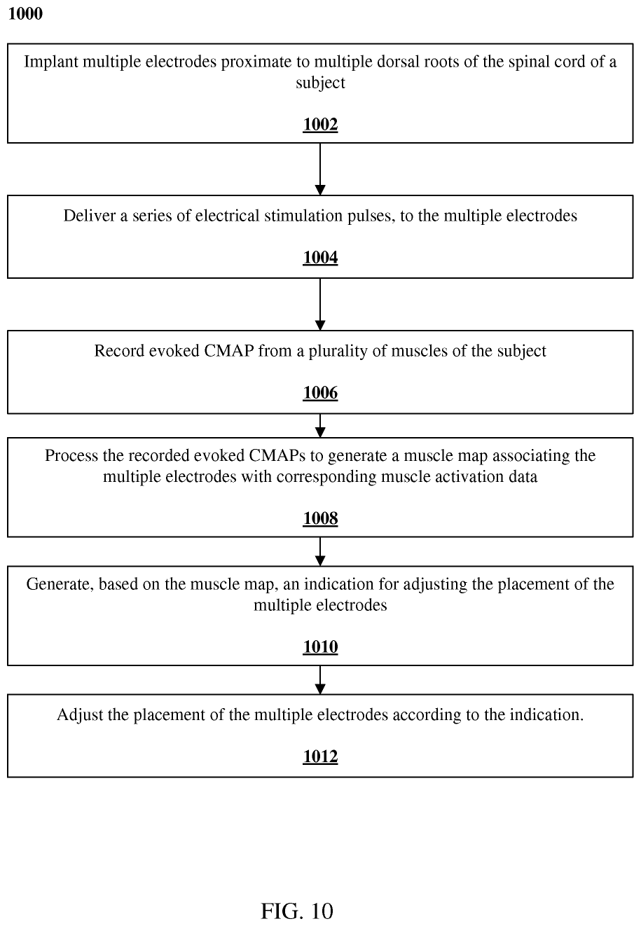

FIG. 10 illustrates a method 1000 for electrophysiology-guided placement of a multi-electrode SCS array. At step 1002, multiple electrodes of an electrode array are implanted proximate to multiple dorsal roots of the spinal cord of a subject, as shown in FIG. 7. At step 1004, a series of electrical stimulation pulses may be delivered to the multiple electrodes, sequentially or simultaneously, via the stimulator 110 or 210. At step 1006, evoked CMAPs are recorded from a plurality of muscles of the subject. At step 1008, the data processor 102 processes the recorded CMAPs to generate a muscle map associating the multiple electrodes with corresponding muscle activation data. At step 1010, the data processor 102 generates, based on the muscle map, an indication for adjusting the placement of the multiple electrodes. The indication may address rostro-caudal positioning (e.g., whether the most rostral electrode activates trapezius at C3-C4 and whether the most caudal electrode activates intrinsic hand muscles at T1), medio-lateral positioning (e.g., whether frequency-dependent suppression is present, indicating dorsal root rather than ventral root activation), and contralateral crossover (e.g., whether CMAPs are detected on the unaffected limb). At step 1012, the placement of the multiple electrodes may be adjusted by the surgeon according to the indication. Steps 1004-1012 may be iteratively repeated until satisfactory placement of the full array is confirmed.

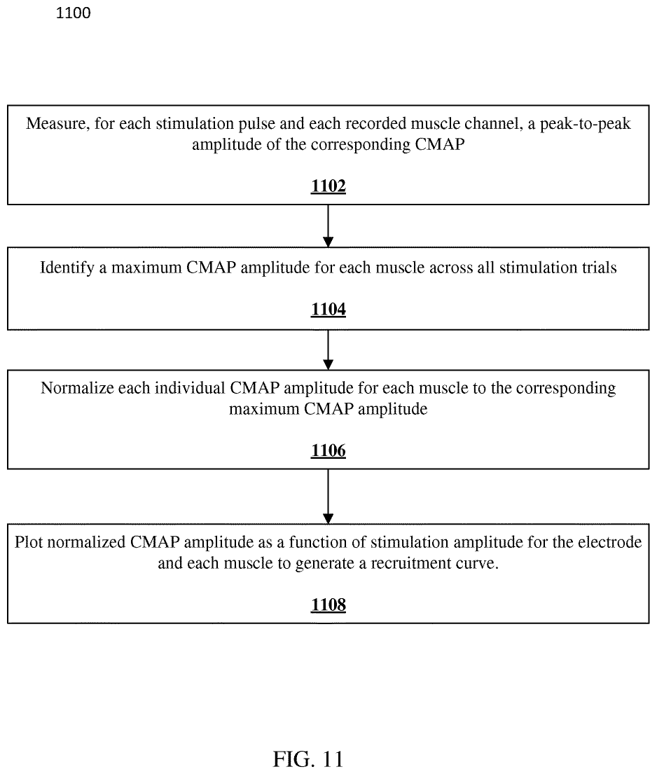

FIG. 11 illustrates a method 1100 for computing and plotting a normalized recruitment curve for a single electrode. At step 1102, for each stimulation pulse delivered to the electrode and for each recorded muscle channel, the peak-to-peak amplitude of the corresponding evoked CMAP may be measured by the data processor 102. At step 1104, the maximum CMAP amplitude for each muscle may be identified across all stimulation trials (i.e., across the full range of stimulation amplitudes tested for that electrode). At step 1106, each individual CMAP amplitude for each muscle may be normalized to the corresponding maximum CMAP amplitude identified at step 1104, yielding normalized values between 0 and 1. At step 1108, the normalized CMAP amplitude may be plotted as a function of stimulation amplitude for the electrode and each muscle to generate a recruitment curve, as illustrated in FIG. 6. Method 1100 represents a per-electrode sub-process of the array-level method 900 (FIG. 9). In method 900, the normalization reference (step 910/912) may be computed across all electrodes in the array to enable cross-electrode comparison; in method 1100 (step 1104), the normalization reference may be computed from the single electrode's own data. Both normalizations are contemplated and may be used depending on the clinical context.

### Examples

### Frequency-Dependent Suppression

FIG. 3 illustrates frequency-dependent suppression data from an exemplary clinical study involving two subjects (SCS01 and SCS02). Each panel shows normalized steady-state CMAP amplitude (y-axis, EMG norm.) as a function of stimulation frequency in Hz (x-axis) for a representative electrode contact at the indicated motor threshold amplitude. For electrode contacts exhibiting transsynaptic dorsal root activation (e.g., SCS02 contacts 2R at 3.3 mA and 1C at 3.0 mA), CMAP amplitude at 0-1 Hz is higher than at 10 Hz and 20 Hz, demonstrating clear frequency-dependent suppression across multiple muscles including anterior deltoid (A Del), biceps (Bic), pronator (Pron), triceps (Tri), wrist flexor (Flx), and wrist extensor (Ext). Contact 7C at 4.2 mA (bottom row) also shows frequency-dependent suppression but with a higher motor threshold, potentially indicative of a more medially positioned electrode. Contact 7R from SCS01 at 1.7 mA shows a low motor threshold with minimal frequency-dependent suppression pattern in most channels except the wrist flexor (Flx), suggesting possible lateral electrode positioning near the dorsal-ventral root convergence zone.

FIG. 4 provides an illustrative time-series EMG trace from SCS02, pronator muscle, electrode contact 1C at 3.0 mA, showing the raw CMAP waveforms at stimulation frequencies of 5 Hz, 10 Hz, and 20 Hz. Stimulation events are indicated by markers above the trace. At 5 Hz, each stimulation pulse produces a high-amplitude, consistent CMAP. At 10 Hz, the CMAP amplitude is visibly reduced compared to the 5 Hz response. At 20 Hz, further amplitude reduction is observed, consistent with progressive synaptic fatigue at the sensory-to-motor synapse. Scale bars of 100 μV (amplitude) and 200 ms (time) are shown.

The data processor 102 automatically detects frequency-dependent suppression using one of two protocols. In a first protocol, stimulation is delivered at a frequency of at least about 20 Hz (40-60 Hz) at a supra-threshold amplitude, and the data processor 102 evaluates the resulting CMAP time-series for a transient initial high-amplitude response decaying to a lower steady-state amplitude over a series of pulses. In a second protocol (used when high-frequency continuous stimulation is not available from the stimulator), stimulation is delivered at a low frequency of less than about 2 Hz, then stepped upward in about 10 Hz increments, with steady-state CMAP amplitude recorded at each frequency. Frequency-dependent suppression is confirmed when steady-state CMAP amplitude decreases monotonically with increasing frequency. If frequency-dependent suppression is not detected, the data processor 102 generates an alert that direct motor neuron activation may be occurring—indicating either that the electrode is placed too laterally near the dorsal-ventral root convergence, or that the lead has migrated ventrally—and recommends corrective lateral repositioning of the lead.

### Stimulation Latency Analysis

FIG. 5 illustrates the measurement of stimulus-to-CMAP latency and its interpretation. The upper panel shows a series of CMAPs (scale 100 μV, 200 ms) recorded following successive stimulation pulses (indicated by blue markers labeled “Stimulation Time” and “Evoked CMAP”). The latency is defined as the time interval from the onset of the stimulation pulse to the onset of the resulting CMAP, measured as the point at which the CMAP amplitude first exceeds a predefined detection threshold above baseline.

The lower panels of FIG. 5 compare two latency profiles: trans-synaptic recruitment (lower left) showing a latency of about 3 ms between the stimulation marker and CMAP onset, and direct recruitment (lower right) showing a latency of less than about 1 ms. Synaptic transmission across a single synapse contributes about 2-3 ms to the total response latency relative to direct axonal conduction. Accordingly, a stimulus-to-CMAP latency of about 3 ms or greater (above the direct conduction time) is indicative of transsynaptic motor neuron recruitment via sensory afferents in the dorsal root. A latency of less than about 1 ms is indicative of direct motor neuron activation.

The data processor 102 automatically measures stimulus-to-CMAP latency for each stimulation pulse and classifies the response as trans-synaptic (latency ≥about 3 ms) or direct (latency <about 1 ms). Where direct activation is detected, the data processor 102 generates an alert and recommends lateral repositioning of the lead or evaluation of adjacent electrodes.

### Recruitment Curves and Muscle Activation Maps

FIG. 6 illustrates recruitment curves and associated anatomical muscle activation maps from an exemplary clinical study for three representative electrode contacts (5R, 2R, 8R). Each recruitment curve plot (top row) shows normalized peak-to-peak CMAP amplitude (y-axis, 0-1) as a function of stimulation current amplitude in mA (x-axis, 0-about 2 mA) for each of eleven muscles, color-coded per the legend (TRAP, A DEL, M DEL, P DEL, BIC, TRI, EXT, FLX, PRO, ABP, ADM). Vertical dashed lines indicate representative clinically relevant stimulation amplitudes (1.4 mA for contact 5R, 1.7 mA for contact 2R, and 1.8 mA for contact 8R). Each recruitment curve follows an S-shaped profile: no response at sub-threshold amplitudes, a rising response above motor threshold, and saturation as all available motor units are recruited.

Below each recruitment curve plot, FIG. 6 shows an anatomical anterior-posterior body diagram for the corresponding electrode contact at the indicated amplitude, with muscles shaded from white (0, no activation) to dark blue (1, maximum activation) according to the normalized CMAP value at that amplitude. A reference color scale bar (normalized peak-to-peak amplitude, 0-1) is provided. The anatomical map for contact 5R at 1.4 mA shows activation concentrated in proximal muscles (deltoid, biceps), consistent with a rostrally-positioned contact. The map for contact 8R at 1.8 mA shows primary activation of distal muscles (wrist flexor, pronator), consistent with a caudally-positioned contact targeting lower cervical segments. The map for contact 2R at 1.7 mA shows intermediate muscle activation patterns.

These recruitment curve plots and anatomical activation maps collectively form the muscle map that may be used by clinicians to select and configure stimulation parameters for specific motor function outcomes during therapy programming.

What is claimed is:

1. A method of adjusting a placement of an electrode of a spinal cord stimulation device in a subject, the method comprising:
implanting the electrode proximate to a dorsal root of a spinal cord;
delivering a series of electrical stimulation pulses, to the electrode;
recording evoked compound muscle action potentials (CMAP) from a plurality of muscles of the subject;
processing the recorded evoked CMAPs to generate a muscle map associating the electrode with corresponding muscle activation data;
generating, based on the muscle map, an indication for adjusting the placement of the electrode; and
adjusting the placement of the electrode according to the indication.

2. The method according to claim 1, further comprising:
determining a motor threshold for the electrode, wherein the motor threshold is a minimum stimulation amplitude sufficient to elicit an identifiable evoked CMAP in at least one of the plurality of muscles; and
generating a notification if a determined motor threshold exceeds a predetermined threshold value.

3. The method according to claim 1, further comprising:
stimulating the electrode at a frequency of between about 20 Hz and about 60 Hz;
detecting a transient change or steady-state reduction in evoked CMAP amplitude as stimulation frequency increases; and
recording, based on the transient change, whether sensory neurons or motor neurons are being directly activated by the stimulation.

4. The method according to claim 1, wherein adjusting the placement of the electrode comprises determining, based on the muscle map, that the electrode should be moved in at least one of a rostral direction, a caudal direction, a medial direction, or a lateral direction.

5. The method according to claim 1, wherein delivering the series of electrical stimulation pulses comprises delivering stimulation pulses sequentially to the electrode at a plurality of stimulation amplitudes ranging from a sub-threshold amplitude to a saturation amplitude.

6. The method according to claim 1, wherein reporting comprises indicating frequency-dependent suppression of CMAP amplitude as evidence that stimulation is activating sensory neurons projecting to spinal motor neurons.

7. The method according to claim 4, further comprising:
measuring, from the recorded CMAP data, a stimulation latency defined as a time elapsed between delivery of a stimulation pulse and onset of a corresponding CMAP waveform; and
classifying, based on the measured stimulation latency, whether the electrical stimulation pulses is directly activating motor neurons or indirectly activating motor neurons via one or more synaptic connections with sensory neurons.

8. The method according to claim 1, further comprising:
placing EMG electrodes on a contralateral, unaffected limb of the subject;
detecting whether CMAP activity is evoked in the contralateral, unaffected limb during stimulation; and
generating, upon detection of contralateral CMAP activity, an indication to move the electrode of the spinal cord stimulation device in a lateral direction away from the spinal midline.

9. The method according to claim 1, further comprising:
transmitting at least one of the muscle map data, recruitment curve data, motor threshold data, and raw EMG data to a cloud-based data storage system; and
retrieving the stored data for use in subsequent stimulation programming.

10. The method according to claim 9, further comprising:
providing the muscle map data and patient-specific programming history data as inputs to a recommendation engine; and
receiving, from the recommendation engine, a suggested indication for adjusting at least one stimulation parameter selected from the group consisting of stimulation amplitude, pulse width, frequency, and electrode selection.

11. The method according to claim 1, further comprising:
implanting multiple electrodes proximate to multiple dorsal roots of the spinal cord of a subject;
delivering a series of electrical stimulation pulses, to the multiple electrodes;
recording evoked CMAP from a plurality of muscles of the subject;
processing the recorded evoked CMAPs to generate a muscle map associating the multiple electrodes with corresponding muscle activation data;
generating, based on the muscle map, an indication for adjusting the placement of the multiple electrodes; and
adjusting the placement of the multiple electrodes according to the indication.

12. The method according to claim 11, wherein automatically processing the recorded CMAPs to generate a muscle map comprises:
measuring, for each stimulation pulse and each recorded muscle channel, a peak-to-peak amplitude of the corresponding CMAP;
identifying a maximum CMAP amplitude for each muscle across all stimulation trials;
normalizing each individual CMAP amplitude for each muscle to the corresponding maximum CMAP amplitude; and
plotting normalized CMAP amplitude as a function of stimulation amplitude for the electrode and each muscle to generate a recruitment curve.

13. The method according to claim 11, further comprising:
computing, for each electrode, a muscle selectivity metric defined as a ratio of normalized CMAP activity of a target muscle to a sum of normalized CMAP activity across all muscles activated by stimulation of that electrode; and
identifying, based on the selectivity metric, a most selective electrode for activating each target muscle.

14. The method according to claim 11, further comprising:
stimulating a most rostral electrode of the multiple electrodes; and determining, based on whether a CMAP is detected in a most proximal target muscle, whether the multiple electrodes require further rostral insertion or caudal repositioning.

15. The method according to claim 11, further comprising:
stimulating a most caudal electrode of the multiple electrodes; and determining, based on whether a CMAP is detected in a most distal target muscle, whether the multiple electrodes require further caudal insertion or rostral repositioning.

16. A method of building a recruitment curve for a spinal cord stimulation electrode array, the method comprising:
delivering, at a first stimulation frequency of between about 1 Hz and 2 Hz, a series of electrical stimulation pulses to a selected electrode of an electrode array, beginning at or below a motor threshold amplitude;
incrementally increasing the stimulation amplitude until a saturation amplitude is reached for at least one muscle of interest or until a predetermined maximum stimulation amplitude is reached;
recording evoked CMAP activity from a plurality of muscles for each stimulation amplitude;
repeating the delivering, incrementally increasing, and recording steps for each electrode in the electrode array;
identifying, for each muscle, a maximum CMAP amplitude across all electrodes and all stimulation amplitudes;
normalizing each individual CMAP amplitude for each muscle using the identified maximum CMAP amplitude; and
generating a recruitment curve plot of normalized CMAP amplitude versus stimulation amplitude for each electrode and each muscle.

17. The method according to claim 16, further comprising:
computing a selectivity index for each electrode-muscle pair; and
generating a suggestion identifying which electrode most selectively activates a given target muscle with minimal co-activation of other muscles.

18. The method according to claim 16, wherein the recording CMAP activity is performed via a multi-channel EMG acquisition system time-synchronized with each stimulation pulse.

19. The method according to claim 18, further comprising:
computing a selectivity index for each electrode-muscle pair; and
generating a suggestion identifying which electrode most selectively activates a given target muscle with minimal co-activation of other muscles.

20. A method of automated stimulation parameter selection for spinal cord stimulation therapy in a patient having post-stroke upper limb motor impairment, the method comprising:
generating, using the method of claim 1, a muscle map associating a plurality of implanted spinal cord stimulation electrodes with corresponding muscles of an affected upper limb;
receiving a desired motor outcome input from a user;
providing the muscle map and the desired motor outcome input to a recommendation engine; and
receiving, from the recommendation engine, a suggested stimulation configuration comprising at least a stimulation electrode selection, a stimulation amplitude, a pulse width, and a stimulation frequency.
